(19) United States
(12) Patent Application Publication
(10) Pub. No.: US 2025/0044503 A1
(43) Pub. Date: Feb. 6, 2025

(54) REDUCED CROSSTALK BETWEEEN CORES OF A MULTICORE FIBER

(71) Applicant: ZSQUARE LTD., Petah Tikva (IL)

(72) Inventors: Asaf SHAHMOON, Petah Tikva (IL); Amihai MEIRI, Ramat Hasharon (IL); Oran HERMAN, Ramat Hasharon (IL); Zeev ZALEVSKY, Rosh HaAyin (IL)

(73) Assignee: ZSQUARE LTD., Petah Tikva (IL)

(21) Appl. No.: 18/924,999

(22) Filed: Oct. 23, 2024

Related U.S. Application Data
(63) Continuation of application No. 17/849,642, filed on Jun. 26, 2022, now abandoned.

(51) Int. Cl. G02B 6/02; A61B 1/00

(52) U.S. Cl. CPC G02B 6/02042; G02B 6/02328; A61B 1/0017

(57) ABSTRACT

Multicore fibers and methods are provided to reduce crosstalk between cores of the multicore fibers. Multicore fibers comprise a plurality of coated and/or cladded cores, which comprise an absorbing and/or scattering coating over a cladding of the core; and/or multilayered cladding comprising at least two cladding layers having different refractive indices. Imaging endoscopes may comprise the multicore fibers and be used for medical imaging.

### CROSS-REFERENCE TO RELATED APPLICATION

The present application is a Continuation of U.S. patent application Ser. No. 17/849,642, filed Jun. 26, 2022, which is hereby incorporated by reference in its entirety.

### BACKGROUND OF THE INVENTION

### 1. Technical Field

The present invention relates to the field of endoscopy, and more particularly, to multicore fiber endoscopes.

### 2. Discussion of Related Art

Multicore fibers include tens or hundreds of thousands of fiber cores and are used to provide high resolution images, e.g., in multicore fiber endoscopes for medical applications. For example, WIPO Publication No. 2019016797, incorporated herein by reference in its entirety, provides various multicore fibers and endoscope configurations. Crosstalk between fiber cores refers to radiation passing from one core to a neighboring core, reducing the quality and accuracy of the resulting image. Reducing crosstalk requires to increase the distance between adjacent cores-which however reduces the resolution of images passed through the multicore fiber and hence its performance.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a multicore fiber comprising a plurality of coated and/or cladded cores, wherein the coated and/or cladded cores comprise at least one of: an absorbing and/or scattering coating over the cores and/or over claddings of the cores; and/or multilayered cladding comprising at least two cladding layers having different refractive indices.

One aspect of the present invention provides a multicore photonic crystal fiber with hollow cores, further comprising a plurality of absorbing and/or scattering rods surrounding the cores, and method of producing thereof.

One aspect of the present invention provides a method comprising reducing or eliminating crosstalk between cores of a multicore fiber comprising a plurality of coated and/or cladded cores, wherein the method comprises at least one of: applying an absorbing and/or scattering coating over claddings of the cores; and/or applying to the cores multilayered cladding comprising at least two cladding layers having different refractive indices.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A, 1B and FIG. 2 are high-level schematic cross section illustrations of multicore fibers, according to some embodiments of the invention.

FIG. 3 is a high-level schematic cross section illustration of photonic crystal multicore fibers, according to some embodiments of the invention.

FIGS. 4A and 4B are high-level schematic flowcharts illustrating methods, according to some embodiments of the invention.

FIGS. 5A-5D are non-limiting examples of disclosed multicore fibers, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Embodiments of the present invention provide efficient and economical methods and mechanisms for reducing crosstalk and/or increasing image quality and resolution of multicore fibers and thereby provide improvements, e.g., to the technological field of medical endoscopy. Multicore fibers and methods are provided to reduce crosstalk between cores of the multicore fibers. Multicore fibers comprise a plurality of coated and/or cladded cores (e.g., tens or hundreds of thousands, or millions of fiber cores), which comprise an absorbing and/or scattering coating over a cladding of the core; and/or multilayered cladding comprising at least two cladding layers having different refractive indices. Imaging endoscopes may comprise the multicore fibers and be used for medical imaging. Crosstalk, or inter-core coupling, refers to radiation exiting from one core and potentially entering another core in the multicore fibers, resulting e.g., from the small diameter of the cores, their high density, due to bending of the fibers, etc. Various embodiments of the invention reduce or eliminate crosstalk in multicore fibers.

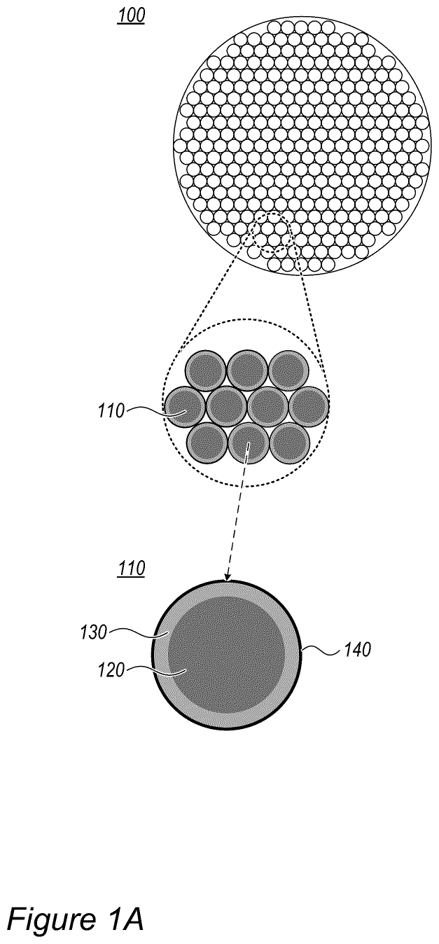

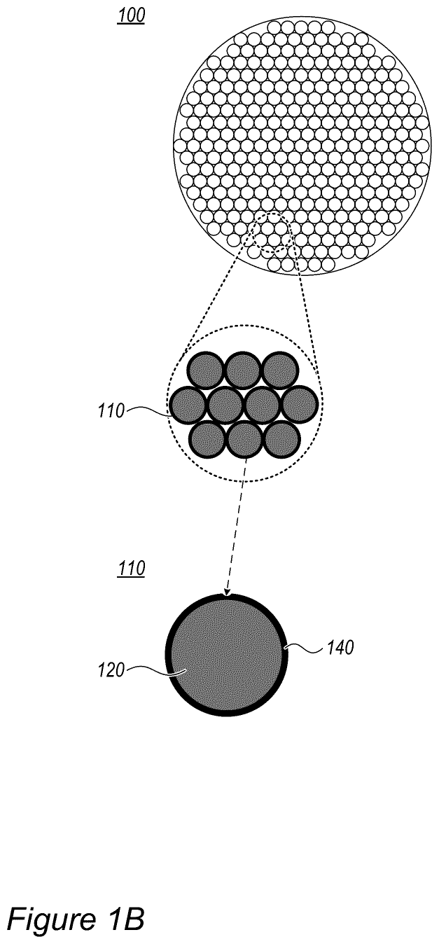

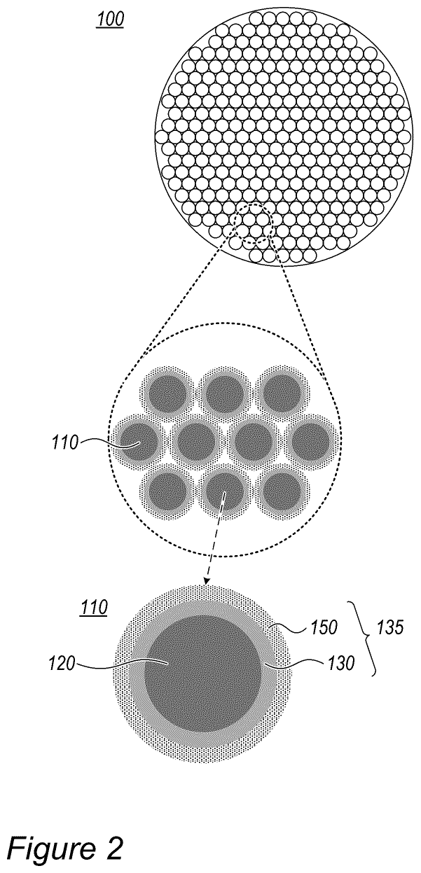

FIGS. 1A, 1B and 2 are high-level schematic cross section illustrations of multicore fiber 100, according to some embodiments of the invention. Multicore fiber 100 may comprise a plurality of coated and/or cladded cores 110. For example, multicore fiber 100 may comprise thousands, tens or hundreds of thousands or millions of coated and/or cladded cores 110 (e.g., at least 1000, 5,000, 10,000, 20,000, 50,000, 100,000, 500,000, 1,000,000, 5,000,000 or any intermediate or higher number of cores) that are coextruded from a preform and have a common outer cladding. Multicore fiber 100 is configured to confine the image radiation passing through the fiber (e.g., from tissue at a distal end of the fiber, upon tissue contact or employing optical elements such as lenses, to an imaging sensor at a proximal end of the fiber) at least mainly to the fiber cores. It is noted that illustrated gaps between coated and/or cladded cores 110 are for clarification only, as in extruded multicore fiber 100 coated and/or cladded cores 110 maybe adjacent without any intermediate gaps. While FIGS. 1A and 1B illustrate schematically coated cores 110 and FIG. 2 illustrates schematically multiply coated cores 110, it is noted that any of disclosed coated and/or cladded cores 110 may be combined within multicore fiber 100.

As illustrated schematically in FIG. 1A, cores 120 may be cladded by a thin cladding 130 which may be coated by an absorbing and/or scattering coating 140 over cladding 130 (it is noted that numeral 110 denoted the cladded cores, including cores 120, their claddings 130 and coatings 140 and/or additional cladding layers as disclosed below). For example, core 120 may have a diameter in the range between 0.3 μm and 7 μm (e.g., 0.5 μm, 1 μm, 2 μm, 3 μm or other intermediate values, or within intermediate ranges such as 0.3-1 μm, 0.5-1.5 μm, 0.8-1.2 μm, 1-2 μm, 2-5 μm, 3-7 μm, etc. or within any other intermediate range) while cladding 130 may be between 0.01 μm and 1 μm thick (e.g., any of 10 nm, 30 nm, 50 nm, 0.1 μm, 0.2 μm, 0.3 μm, 0.5 μm, 1 μm or intermediate values).

As illustrated schematically in FIG. 1B, cores 120 may be coated by absorbing and/or scattering coating 140 over cladding 130 and/or directly over cores 120. Coating 140 may be absorbing (e.g., black) and/or scattering (e.g., metallic) and be configured to reduce or eliminate crosstalk between different cores 120. For example, absorbing coatings 140 may be configured to reduce or eliminate radiation exiting cores 120 (and penetrating cladding 130 if present) by absorption of the exiting radiation. In another example, scattering coatings 140 may be configured to reduce or eliminate radiation exiting cores 120 (and penetrating cladding 130 if present) by reflecting of the exiting radiation back into cores 120 (or cladding 130). Coating 140 may be between 50 nm and 100 nm thick, e.g., any of 50 nm, 60 nm, 70 nm, 70 nm, 80 nm, 90 nm, 100 nm or intermediate values. Non-limiting examples for coating materials include reflective metals such as tin, copper, gold or aluminum and/or dark or black materials such as various black dyes (e.g., carbon-based) applied directly onto cladding 130 or mixed with polymer material such as ethylene methyl acrylate (EMA) or tetrafluoroethylene-hexafluoropropylene-vinylidene fluoride (THV polymer), or other coating material with a low refractive index. For example, cladded cores 110 may comprise cores 120 made of styrene methyl methacrylate (SMMA), cladding 130 made of polymethyl methacrylate (PMMA), and coating 140 made of EMA and/or THV polymer with added black dye.

As illustrated schematically in FIG. 2, cores 120 may be cladded by a multilayered cladding 135 which may comprise two or more layers of cladding 130, 150 configured to reduce crosstalk among cores 120. For example, core 120 may have a diameter of 1 μm (or, e.g., in a range of between 0.5 μm and 1.5 μm, or in a range of between 0.8 μm and 1.2 μm, or within any other intermediate range) while multilayered cladding 135 may be between 0.1 μm and 5 μm thick (e.g., any of 0.1 μm, 0.5 μm, 0.7 μm, 1 μm, 1.5 μm, 2 μm, 3 μm, 4 μm, 5 μm, or intermediate values). For example, each of claddings 130, 150 may be between 0.01 μm and 1 μm thick (e.g., any of 10 nm, 30 nm, 50 nm, 0.1 μm, 0.2 μm, 0.3 μm, 0.5 μm, 1 μm or intermediate values). In case of two-layered multilayered cladding 135, with cores 120 having a refractive index of 1.5 as a non-limiting example, inner cladding 130 may have a refractive index of between 1.3-1.7 (e.g., any of 1.3, 1.4, 1.5, 1.6, 1.7 or intermediate values) while outer cladding 150 may have a refractive index of between 1.0-1.5 (e.g., any of 1.0, 1.1, 1.2, 1.3, 1.4, 1.5, or intermediate values). The multiple layers of cladding may be configured to enhance reflection of radiation back into core 120 and/or possible absorption of radiation exiting core 120, to reduce or eliminate crosstalk between cores 120 (e.g., ensuring total internal reflection). It is noted that while multilayered cladding 135 provide smaller differences in refractive indices between layers than using air gaps between cores 120, the multiple reduction steps of the refractive index between consecutive cladding layers may likewise reduce overall crosstalk between cores by reducing the numerical aperture (NA=\sqrt{n_{core}²−n_{cladding}²}=n·sin θₘₐₓ; θₘₐₓ being the maximal angle still maintaining total internal reflection) of the radiation mode that interfaces the cladding, so that the mode has less interaction with adjacent cores, and over a larger range of fiber bending angles (as the possible bending angle is inversely proportional to the NA).

Multicore fibers 100 of any of the disclosed embodiments may be fabricated from a preform in which the rods that are drawn (pulled) to form cores 110—are coated in the preform by a corresponding absorbing and/or scattering coating, and/or by a multilayered cladding. For example, core-forming rods may be directly coated by an absorbing and/or scattering coating and drawn to form multicore fibers 100 as illustrated, e.g., in FIG. 1B, cladded core-forming rods may be coated (on the cladding) by an absorbing and/or scattering coating and drawn to form multicore fibers 100 as illustrated, e.g., in FIG. 1A, and/or the rods may be cladded by two or more layers with corresponding refractive indices and drawn to form multicore fibers 100 as illustrated, e.g., in FIG. 2.

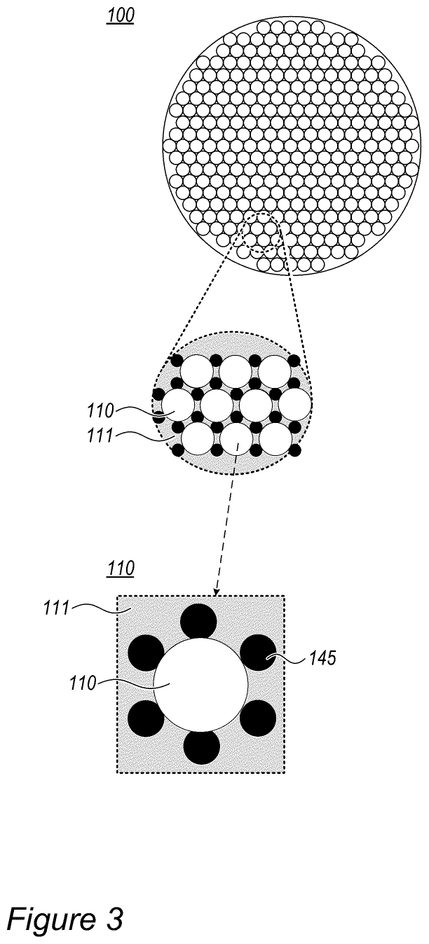

FIG. 3 is a high-level schematic cross section illustration of photonic crystal multicore fibers 100, according to some embodiments of the invention. Multicore fibers 100 may be configured as photonic crystal fibers (PCFs) 100 in which cores 110 are hollow (e.g., air-filled) and periodic over the cross section of PCF 100, surrounded by fiber material 111 (e.g., silica glass or polymer material). Multicore PCFs 100 may comprise a plurality of absorbing and/or scattering rods 145 surrounding cores 110 and preventing crosstalk between cores 110. Light guiding cores 110 may be surrounded by periodic structures of light absorbing rods 145 to prevent crosstalk, in addition or in place of coating 140. Non-limiting examples for fiber material include polymeric material such as: polymethyl methacrylate (PMMA), polystyrene (PS), Styrene Methyl Methacrylate (SMMA), tetrafluoroethylene-hexafluoropropylene-vinylidene fluoride (THV polymer), etc. In photonic crystal multicore fibers 100, cores 120 may have a diameter between 0.3 μm and 7 μm (e.g., 0.5 μm, 1 μm, 2 μm, 3 μm or other intermediate values, or within intermediate ranges such as 0.3-1 μm, 0.5-1.5 μm, 0.8-1.2 μm, 1-2 μm, 2-5 μm, 3-7 μm, etc. or within any other intermediate range), spaced by fiber polymer material that may be between 0.01 μm and 1 μm thick (e.g., any of 10 nm, 30 nm, 50 nm, 0.1 μm, 0.2 μm, 0.3 μm, 0.5 μm, 1 μm or intermediate values).

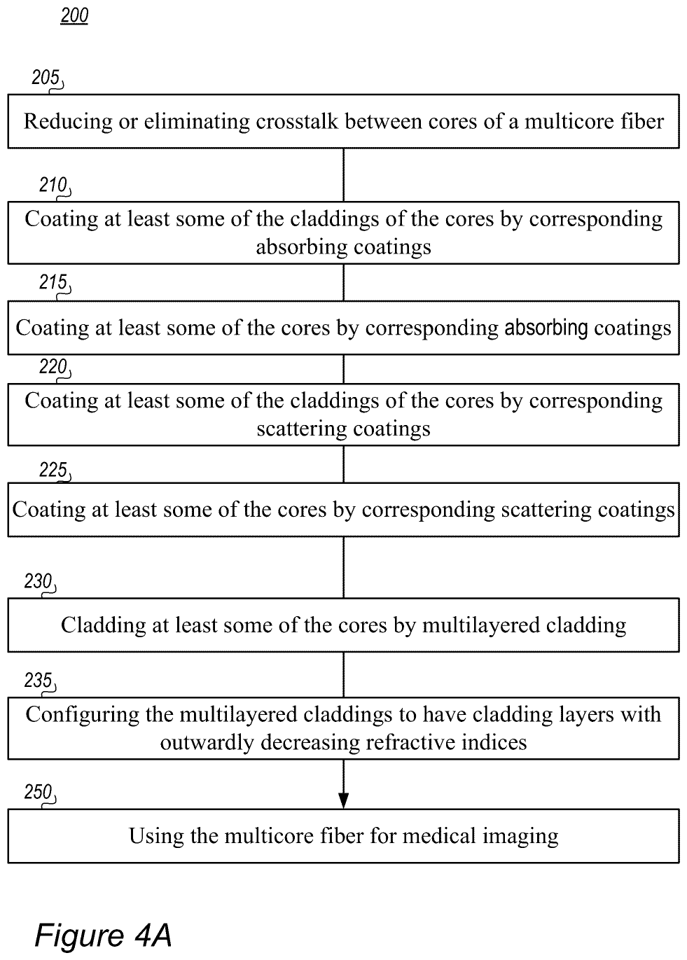

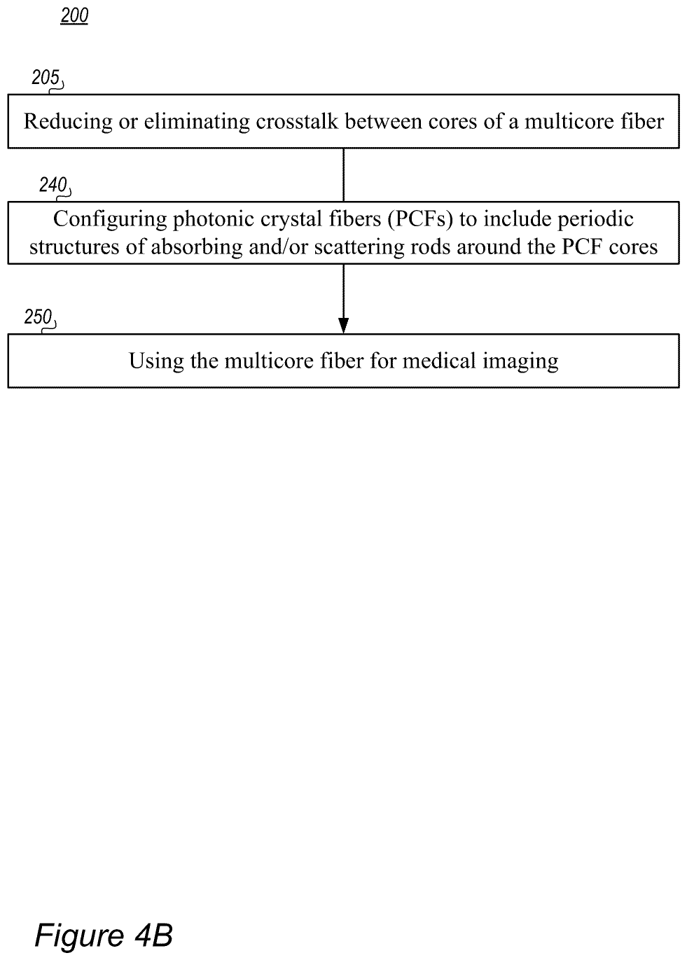

FIGS. 4A and 4B are high-level schematic flowcharts illustrating methods 200, according to some embodiments of the invention. The method stages may be carried out with respect to multicore fiber 100 described above, which may optionally be configured to implement method 200. Method 200 may comprise the following stages, irrespective of their order.

As illustrated schematically in FIG. 4A, method 200 comprises reducing or eliminating crosstalk between cores of a multicore fiber (stage 205) comprising a plurality of coated and/or cladded cores (thousands or tens of thousands of cores 110, e.g., at least 1000, 5,000, 10,000, 20,000 or any intermediate or higher number of cores). Method 200 comprises applying an absorbing and/or scattering coating over claddings of the cores (stage 210, 220) and/or directly over unclad cores (stage 215, 225); and/or applying to the cores multilayered cladding comprising at least two cladding layers having different refractive indices (stage 230). Method 200 may further comprise using the multicore fiber for medical imaging (stage 240). The coating and/or cladding processes may be applied in a preform used to prepare the multicore fiber.

For example, method 200 may comprise coating at least some of the claddings of the cores by corresponding absorbing (e.g., black) coatings (stage 210) and/or coating at least some of the claddings of the cores by corresponding scattering (e.g., metallic) coatings (stage 220). Alternatively or complementarily, method 200 may comprise coating at least some of the cores by corresponding absorbing coatings (stage 215) and/or coating at least some of the cores by corresponding scattering coatings (stage 225). Thinly cladded cores (e.g., 0.5 μm-1.5 μm wide cores cladded by 0.01 μm-2 μm thick cladding) and/or unclad cores may be coated by a scattering and/or an absorbing coating that may be between 50 nm and 100 nm thick. Any of the coating processes may be carried out by coating the rods of the preform that are drawn to form the multicore fiber. The rods (bare or cladded) may be coated before they are used to form the preform. The absorbing coating may be configured to reduce or eliminate crosstalk between the cores by absorbing radiation that may penetrate the thin cladding (e.g., ensuring total internal reflection).

In another example, method 200 may comprise cladding at least some of the cores by multilayered cladding (stage 230), e.g., configured to have cladding layers with outwardly decreasing refractive indices (stage 235). The multilayered cladding (cladding, e.g., 0.5 μm-1.5 μm wide cores) may include two or more layers (e.g., each 0.2 μm-2 μm thick) having outwardly decreasing refractive indices (e.g., between 1.3-1.7 for the innermost cladding layer decreasing to 1.0-1.5 for the outermost cladding layer). Cladding materials may comprise any type of polymer with corresponding refractive indices, such as PMMA, PS, SMMA, THV, etc.

As illustrated schematically in FIG. 4B, method 200 may comprise configuring the multicore fibers as photonic crystal fibers (PCFs) to include periodic structures of absorbing and/or scattering rods around the PCF cores. These periodic structures may be configured to reduce or prevent crosstalk between the cores.

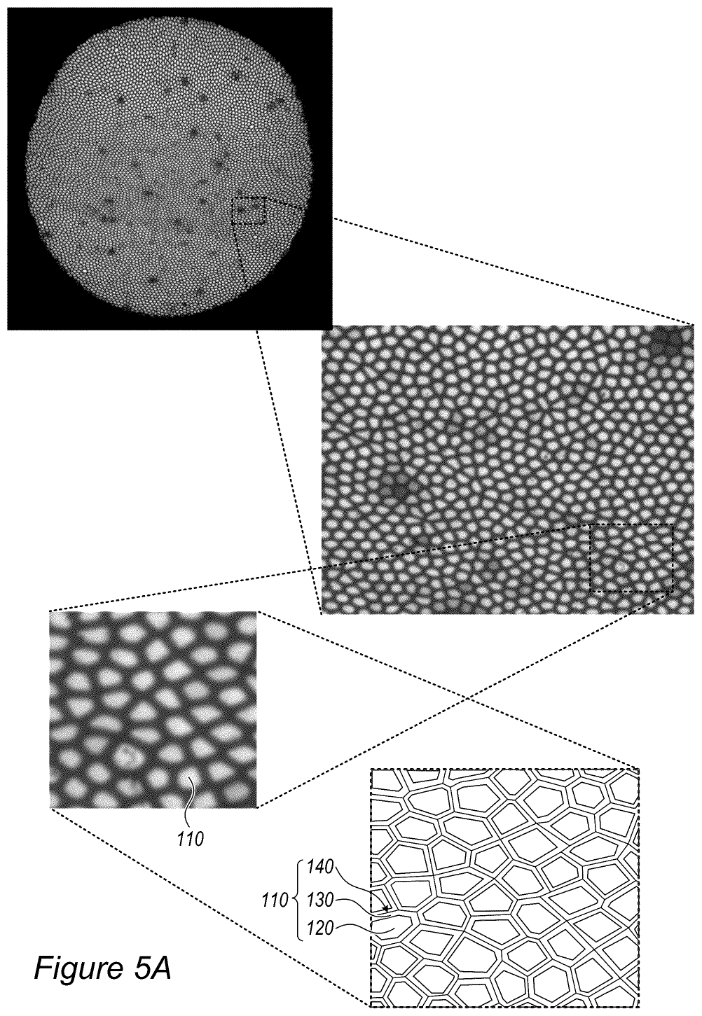

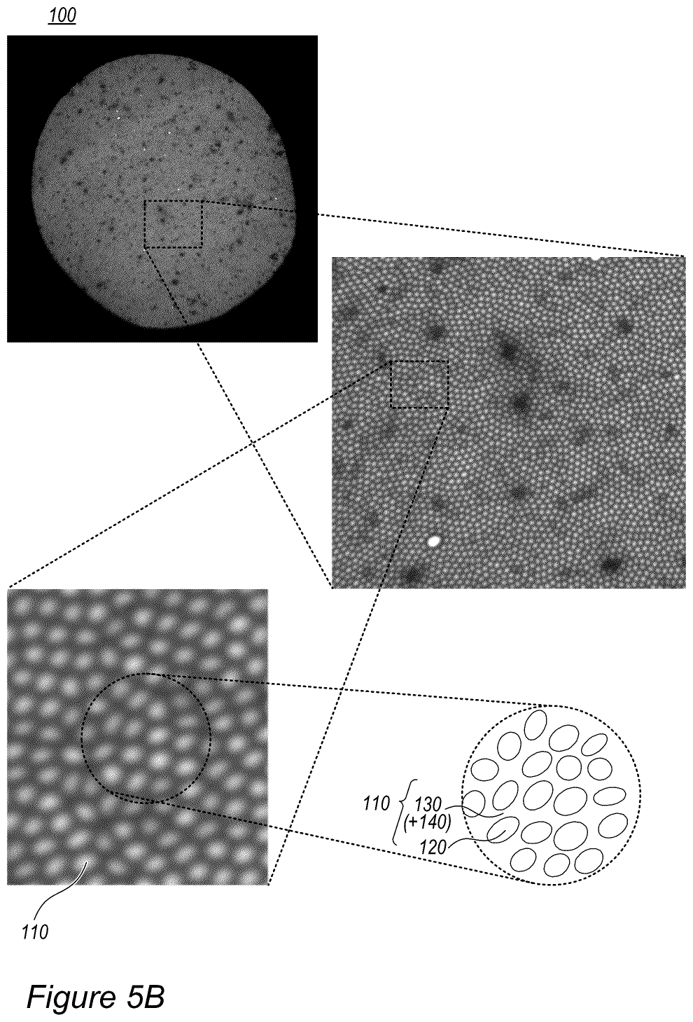

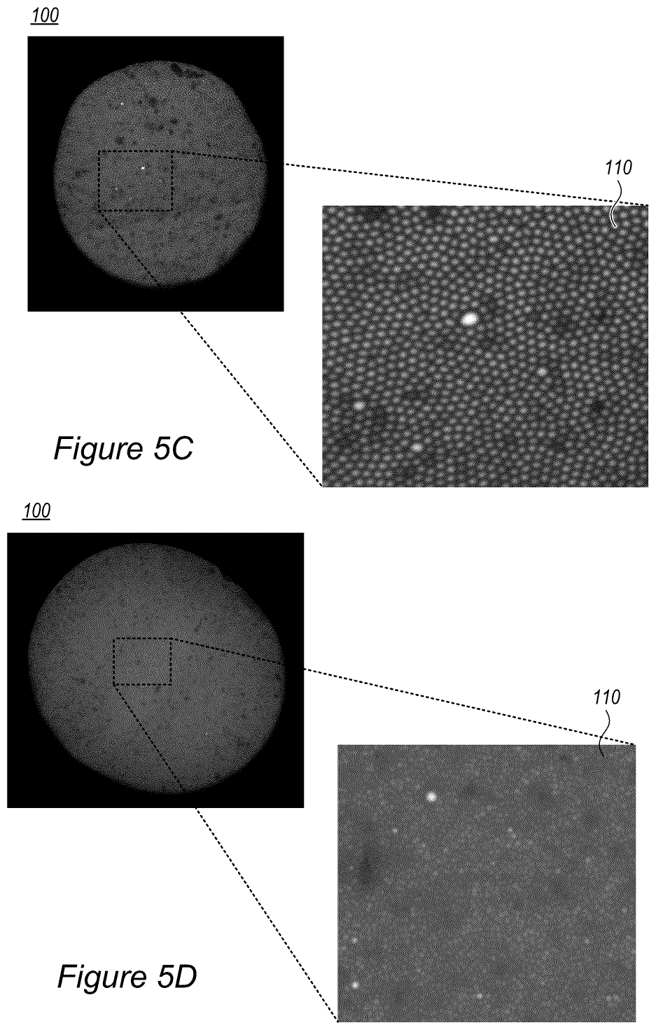

FIGS. 5A-5D are non-limiting examples of disclosed multicore fibers 100, according to some embodiments of the invention. The denoted enlarged regions in consecutive images are approximate. FIG. 5A illustrated fiber 100 with an outer diameter of 640 μm, with approximately 7,500 cladded cores 110 having cores 120 made of SMMA, having a diameter of 5.3 μm and at a pitch of 7 μm, with cladding 130 made of PMMA, and coating 140. FIG. 5B illustrated fiber 100 with an outer diameter of 640 μm, with approximately 30,000 cladded cores 110 having cores 120 made of SMMA, having a diameter of 2.5 μm and at a pitch (distance between core centers) of 3.6 μm, with cladding 130 made of PMMA, and coating 140. FIG. 5C illustrated fiber 100 with an outer diameter of 320 μm, with approximately 7,500 cladded cores 110 having cores 120 made of SMMA, having a diameter of 2.5 μm and at a pitch of 3.6 μm, with cladding 130 made of PMMA, and coating 140. FIG. 5D illustrated fiber 100 with an outer diameter of 640 μm, with approximately 120,000 cladded cores 110 having cores 120 made of SMMA, having a diameter of 0.9 μm and at a pitch of 1.8 μm, with cladding 130 made of PMMA, and coating 140. It is noted that the indications of coating 140 relate to its location but not necessarily to its implementation.

Elements from FIGS. 1A-4B may be combined in any operable combination, and the illustration of certain elements in certain figures and not in others merely serves an explanatory purpose and is non-limiting.

Advantageously, disclosed multicore fibers 100 may be used for high resolution imaging, maintain small distances between cores and high core density while reducing or eliminating inter-core crosstalk. It is noted that in contrast to multilayered fibers that are designed as photonic crystals, disclosed multicore fibers 100 do not necessarily have spatial periodicity, as the applied coatings and/or multilayered cladding rather than spatial periodicity are utilized to prevent crosstalk.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A multicore fiber comprising a plurality of coated or cladded cores, wherein the coated or cladded cores comprise at least one of:
an absorbing or scattering coating over the core or over a cladding of the core; and
multilayered cladding comprising at least two cladding layers having different refractive indices.

2. The multicore fiber of claim 1, wherein the absorbing or scattering coating is between 50 nm and 100 nm thick.

3. The multicore fiber of claim 1, wherein at least some of the cores comprise a black coating over the cladding of the cores.

4. The multicore fiber of claim 3, wherein the cladding is between 0.01 μm and 1 μm thick and the coating is absorbing.

5. The multicore fiber of claim 3, wherein the cladding is between 0.01 μm and 1 μm thick and the coating is scattering.

6. The multicore fiber of claim 1, wherein at least some of the cores comprise a black coating over the cores.

7. The multicore fiber of claim 1, wherein at least some of the cores comprise a metallic coating over the cores.

8. The multicore fiber of claim 1, wherein at least some of the cores comprise multilayered cladding.

9. The multicore fiber of claim 8, wherein the multilayered cladding is between 0.2 μm and 5 μm thick.

10. The multicore fiber of claim 8, wherein the multilayered cladding comprises two cladding layers having refractive indices of between 1.3-1.7 and 1.0-1.5, respectively.

11. The multicore fiber of claim 8, wherein the multilayered cladding comprises two cladding layers having refractive indices of between 1.3-1.5 and 1.1-1.3, respectively.

12. The multicore fiber of claim 1, comprising cores made of styrene methyl methacrylate (SMMA), cladded by polymethyl methacrylate (PMMA) and coated by tetrafluoroethylene-hexafluoropropylene-vinylidene fluoride (THV polymer) and/or ethylene methyl acrylate (EMA) with black dye.

13. The multicore fiber of claim 1, comprising cores made of styrene methyl methacrylate (SMMA), cladded by polymethyl methacrylate (PMMA) and coated by tin, copper, gold or aluminum.

14. An imaging endoscope comprising the multicore fiber of claim 1.

15. A multicore photonic crystal fiber with hollow cores, further comprising a plurality of absorbing or scattering rods surrounding the cores.

16. The multicore photonic crystal fiber of claim 15, wherein the absorbing or scattering rods form periodic structures surrounding the cores.

17. A method comprising reducing or eliminating crosstalk between cores of a multicore fiber comprising a plurality of coated or cladded cores, wherein the method comprises at least one of:
applying an absorbing and/or scattering coating over claddings of the cores; and/or
applying to the cores multilayered cladding comprising at least two cladding layers having different refractive indices.

18. The method of claim 17, comprising coating at least some of the claddings of the cores by absorbing coatings.

19. The method of claim 17, comprising coating at least some of the claddings of the cores by scattering coatings.

20. The method of claim 17, comprising cladding at least some of the cores by multilayered claddings.

21. The method of claim 20, further comprising configuring the cladding layers to have outwardly decreasing refractive indices.
